(19) United States
(12) Patent Application Publication
(10) Pub. No.: US 2005/0058699 A1
(43) Pub. Date: Mar. 17, 2005

(54) Sterile preparations of phospholipids and anti-inflammatory pharmaceuticals and methods for making and using same

(75) Inventors: Lenard M. Lichtenberger, Houston, TX (US); Elizabeth J. Dial, Houston, TX (US)

Correspondence Address: ROBERT W STROZIER, P.L.L.C, PO BOX 429, BELLAIRE, TX, 77402-0429, US

(73) Assignee: THE BOARD OF REGENTS OF THE UNIVERSITY OF TEXAS SYSTEM

(21) Appl. No.: 10/909,748

(22) Filed: Aug. 2, 2004

Related U.S. Application Data
(60) Provisional application No. 60/491,568, filed on Jul. 31, 2003.

(51) Int. Cl. A61K 9/127

(52) U.S. Cl. 424/450

(57) ABSTRACT

A filter sterilized composition of a phospholipid and an anti-inflammatory pharmaceutical is disclosed, where the anti-inflammatory pharmaceutical is a nonsteroidal, anti-inflammatory drug (NSAID), a cyclooxygenase 2 (COX-2) inhibitor or a mixture thereof. A method for preparing these sterile compositions is also disclosed and includes a filtration step through a sterilizing filtration membrane. Methods for using these sterilized compositions to treat accident and battle field injuries or treatment of injuries to the nerve system especially in unconscious patients via injection, topical administration, or according to an administration protocol.

### RELATED APPLICATIONS

This application claims provisional priority to U.S. Provisional Application Ser. No. 60/491,568, filed Jul. 31, 2003.

### BACKGROUND OF THE INVENTION

1. Field of the Invention

The present invention relates to sterile composition including a phospholipid (PL) and an anti-inflammatory pharmaceutical (AIP) such as a nonsteroidal, anti-inflammatory drugs (NSAID), a cyclooxygenase 2 (COX-2) inhibitor or the like or mixtures or combinations thereof and methods for making and using same, where the preparations are capable of passing through a filter having a pore size sufficiently small to result in a filtrate that is considered sterile for medical applications.

More particularly, the present invention relates to a membrane-filterable, sterile, PL-AIP composition including a phospholipid (PL) and an anti-inflammatory pharmaceutical (AIP), where the AIP include an NSAID, COX-2 inhibitor, or the like, or mixtures or combinations thereof, and where preferably, the PC-AIP composition is an association complex of the PL and the AIP. The present invention also relates to methods for making the sterile preparations, where the methods include the step of adding an anti-inflammatory pharmaceutical to an aqueous composition comprising phospholipid liposomes and/or phospholipid micelles to form a filterable composition considered sterile for medical applications. The present invention also relates to methods for using the sterile compositions, where the methods include administering the sterile compositions either orally, topically, intravenously, intra-arterially or directly into a tissue site of an animal including a human to ameliorate inflammation, pain, fever or other symptoms for which NSAIDs and COX-2 inhibitors are known to ameliorate.

2. Description of the Related Art

For a background of phospholipids and anti-inflammatory pharmaceuticals the reader is directed to U.S. Pat. Nos. 4,918,063; 5,043,329; 4,950,656; 5,032,585; 5,763,422; and 5,955,451 and PCT/JUS01/51605, incorporated herein by reference.

In postoperative pain management, health care professionals generally are required to administer opioids, other potent analgesics, or both. Although these medications have been proven pain management properties, they also have a significant number of potential side effects, including nausea, vomiting, constipation, pruritus, urinary retention, respiratory depression, and sedation. Although nonsteroidal, anti-inflammatory drugs (NSAIDs) provide anti-inflammatory and analgesic effects, they are limited to oral or rectal administrations greatly limiting the use of NSAIDs under postoperative conditions. Currently, ketorolac tromethamine is only NSAID that, can be administered intravenously, intramuscularly, or orally.

Thus, there is a need in the art for improved sterile preparations of a wider range of anti-inflammatory pharmaceuticals combined with phospholipids so that the anti-inflammatory benefits of the anti-inflammatory pharmaceuticals can be experienced without the concurrent damage to hydrophobic membranes and/or layers or can be administered internally because the compositions are sterile.

### SUMMARY OF THE INVENTION

The present invention provides sterile compositions including a phospholipid (PL) and anti-inflammatory pharmaceutical (AIP), where the AIP includes an NSAID, COX-2 inhibitor or the like, or mixtures or combinations thereof, where the compositions are sterile filterable at a pH range sufficient to effectuate filtration and the filter has a pore size sufficiently small to form a PL-AIP composition considered to be sterile for medical applications. The compositions of this invention can include one or more phospholipids and one or more anti-inflammatory pharmaceuticals, i.e., compositions including one or more phospholipids and a single anti-inflammatory pharmaceutical, compositions including a single phospholipid and one or more anti-inflammatory pharmaceutical or compositions including one or more phospholipids and one or more anti-inflammatory pharmaceuticals. Such compositions can be mixtures of separately prepared PL-AIP compositions or composition including one or more phospholipid and/or one or more an anti-inflammatory pharmaceutical; provided that a pH range exists that facilitates passage of the compositions through the sterilizing filter to form compositions considered sterile for medical application, especially, pain management where the compositions are directly injected into an animals including a human body.

The present invention provides sterile compositions ofphospholipids and anti-inflammatory pharmaceuticals including NSAIDs, COX-2 inhibitors or the like, or mixtures or combinations thereof, where the compositions are sterile filterable at a pH range sufficient to permit the composition to pass through the filter forming a medicinally sterile composition.

The present invention provides method for making a sterile compositions including the steps of contacting an aqueous composition including a phospholipid and an anti-inflammatory pharmaceutical under agitating conditions at a operable pH range to form an agitated phospholipid/anti-inflammatory pharmaceutical (PL-AIP) preparation, where the operable pH range permits the PL-AIP preparation to pass through a sterilizing filter. The method also includes the step of passing the agitated PL-AIP preparation through a filter such as a membrane filter having a pore size sufficiently small to produce a filter sterilized PL-AIP composition for use in medical application requiring an effective amount of an pain management composition to be injected directly into a body of an animal including a human.

The present invention provides methods for administering a pharmaceutically effective amount of a filter sterilized phospholipid/anti-inflammatory pharmaceutical (PL-AIP) composition including the steps of orally administering, topically administering, intravenously administering, intra-arterially administering or directly administering into a tissue site an effective amount of a composition of this invention, where the administration can be a single administration, a periodic administration, a intermittent administration, or administration according to any administration protocol, which can include one or more oral, topical, intravenous, intra-arterial, directly into a tissue site administration or combinations of these administration formats.

The present invention provides methods of treating spinal cord injuries, traumatic brain injuries, strokes, injuries to the peripheral nerves system, injuries to the central nerves systems or injuries to other systems having nerve tissue, preferably the injury has associated with it inflammation, where the methods include the step of administering a composition of this invention to an animal including a human or directly to the site of injury or into the blood or other bodily fluid of the animal including a human.

The present invention provides methods of treating field injuries such as combat injuries or accident injuries, where the methods include the steps of administering an amount of a composition of this invention directly to the injury or to the surrounding tissue to reduce inflammation while preventing ulceration of the injury or while maintaining the integrity of hydrophobic membranes and/or layers that may be associated with the injured site, where the amount of the composition administered is sufficient to cause a desired pharmacological effect.

The present invention provides a method for preparing a sterile filtration formulation including aphospholipid such as phosphatidylcholine (PC) and an anti-inflammatory pharmaceutical such as a nonsteroidal, anti-inflammatory drug (NSAID), a COX-2 inhibitor, or a mixture or combination thereof, where the method includes the step of drying a solvent solution including a phospholipid to form a phospholipid film. The phospholipid film is then resuspended in a solution of an NSAID, a COX-2 inhibitor, or a mixture or combination thereof, under agitation such as sonication or other equivalent agitation techniques, where the solution is maintained at a pH near a pKₐ of the NSAID to form an aqueous composition including PC-NSAID liposomes or micelles. The aqueous composition is then passed or extruded through a filter having a pore size sufficiently small to produce a sterile filtered composition, where the pore size is about 0.22 μm or less. The resulting sterile filtered composition (an aqueous solution) is adjusted to physiological pH making it an injectable suitable for via intravenous injection, intra-arterial injection, intramuscular injection, injection directly into a tissue site or injection directly into an injury site. The compositions are ideally suited for post operative administration to reduce inflammation, pain and other post operative symptoms via direct injection into the body such as intravenous, intra-arterial or direct injection into the affected tissue. The sterile compositions can be used in wound dressings, in wound ointments, or in any other material that can be administered directly to a wound in the field, especially under battle field conditions.

The present invention also provide an injection apparatus including a reservoir including a volume of a composition of this invention sufficient to cause a desired pharmacological effect, a plunger operably connected to the reservoir and a needle operably connected to an other end of the reservoir, where the volume is injected through the needle when the plunger is depressed.

The present invention also provide a kit for emergency administration of a sterile injectable pain relieving PL-AIP compositions, where the kit includes an injector apparatus including a manual or electrically powered syringe, a needleless injection system or other apparatus that can inject the composition into a body of an animal including a human. The kit also includes containers including doses of at least one PL-AIP composition sufficient to cause desired pharmacologic effects.

### Definitions

Unless otherwise stated, the following terms shall have the following meanings:

The term “fluid” means a liquid and any mixture of a liquid and a solid that has fluid attributes, e.g., flowable or having appreciable fluidity a standard temperature and pressure, including, without limitation, a dispersion of a solid(s) in a liquid, an emulsion, a slurry, a micro-emulsion, colloidal suspension, a suspension, a suspension of liposomes, a suspension of micelles or the like.

The term “molecular association or associated complex” means a combination of two or more molecular species associated via any known stabilizing atomic or molecular level interaction or any combination thereof, where the interactions include, without limitation, bonding interactions such as covalent bonding, ionic bonding, hydrogen bonding, coordinate bonding, or any other molecular bonding interaction, electrostatic interactions, a polar or hydrophobic interactions, or any other classical or quantum mechanical stabilizing atomic or molecular interaction.

The term “liposome” is defined as small, artificially-created spheres whose walls are phospholipid bilayers. They are made by mixing dry phospholipids, such as egg yolk, in water. The lipid bilayer can fuse with the lipids in cell membranes, so liposomes hold much promise as agents for delivering drugs or other chemicals directly into cells. Liposomes generally are spherical particles having a diameter between about 100 and about 2000 nm.

The term “micelle” is defined as a colloidal aggregate of amphipathic (surfactant) molecules, which occurs at a well-defined concentration known as the critical micelle concentration. The typical number of aggregated molecules in a micelle (aggregation number) is 50 to 100. Micelles generally are spherical particles having a diameter between about 2 and about 10 mm.

The term “animal” is defined as any species in the animal kingdom including mammals.

The term “mammal” is defined as any class of warm-blooded higher vertebrates that includes humans.

The term “phospholipid” refers any lipid or fatty acid having a covalently attached a phosphate group in the molecular structure.

The term “zwitterionic phospholipid” means a phospholipid having a proton acceptor in the molecular structure so that the phosphate group can bear a negative charge and the proton acceptor can be a positive charge due to an intra-molecular acid-base reaction.

The term “heterocyclyl” means a saturated or unsaturated 5 to 7-membered heterocyclic group with one or two rings and 1 to 3 heteroatoms, independently chosen from N, O or S.

The term “aryl” denotes a substituted or unsubstituted phenyl, furyl, thienyl or pyridyl group, or a fused ring system of any of these groups, such as naphtyl.

The term “substituted aryl” denotes an aryl group as defined above which is substituted by one or more alkyl, alkoxy, halogen, amino, thiol, nitro, hydroxy, acyl. aryl or cyano groups.

The term “colloidal metal” denotes any metal or metal-containing compound that can be formed into a colloidal suspension or dispersion.

The term “metal complex” denotes complexes of any metal classified as such in the Periodic Chart of Elements and preferably, complexes of non-alkali metals.

The term “polyvalent metal complex” denotes any complex of a metal, where the metal can have, carry or bear a positive charge greater than 1 and generally from 2 to 6.

The term “zwitterion” denotes a molecule having both a positive charged group and a negatively charged group.

The term “zwitterionic form” denotes a molecule that has a positive charged group and a negatively charged group. Generally, the reaction conditions are adjusted so that intramolecular hydrogen ion transfer can occur.

The term “pharmaceutically effective amount” denotes an amount of NSAD required to cause a measurable reduction in an NSAID affected symptoms such as pain reduction, fever reduction, inflammation reduction, or the like.

The term “sterile filtrate” means a preparation that has passed through a filter having a pore size sufficiently small to result in a preparation free or substantially free of bacterial contaminants. Bacteria generally range in size from about 0.2 μm to about 600 μm, with most bacteria having a size in the range of about 1 μm to about 10 μm. Filters having pore size of about 0.22 μm or less are considered to produce sterile filtrates and are sufficiently small to result in a filter sterilized composition. Such filters and filter kits are available from Millipore Corporate, 290 Concord Rd., Billerica, Mass. 01821 USA as well as other manufacturers.

### DESCRIPTION OF THE DRAWINGS

The invention can be better understood with reference to the following detailed description together with the appended illustrative drawings in which like elements are numbered the same:

FIG. 1 depicts a graph of filtration sterilization of dipalmitoyl phosphatidylcholine (DPPC) liposomes with or without indomethacin (INDO) pH 8 after 10 minutes of sonication;

FIG. 2 depicts a graph of filtration sterilization of DPPC preparations with or without indomethacin and ibuprofen at pH 8 after 10 minutes of sonication;

FIG. 3 depicts a graph of filtration sterilization of DPPC preparations with or without ibuprofen at pH values between 5 and 8 after 10 minutes of sonication;

FIG. 4 depicts a graph of filtration sterilization of DPPC preparations with or without ibuprofen at pH values between 5 and 8 after 20 minutes of sonication;

FIG. 5 depicts a graph of filtration sterilization of DPPC preparations with or without ibuprofen at pH 6 at different DPPC:ibuprofen ratios holding the ibuprofen concentration fixed after 10 minutes of sonication;

FIG. 6 depicts a graph of filtration sterilization of DPPC preparations with or without ibuprofen at pH 6 at different DPPC:ibuprofen ratios holding the DPPC concentration fixed after 10 minutes of sonication;

FIG. 7 depicts a graph of filtration sterilization of DPPC preparations with or without aspirin (ASA) and indomethacin (INDO) at pH 8 after 20 minutes of sonication; and

FIG. 8 depicts a graph of filtration sterilization of DPPC preparations with or without aspirin (ASA) at a pH between 3 and 8 after 20 minutes of sonication.

### DETAILED DESCRIPTION OF THE INVENTION

The inventors have found that compositions of phospholipids that generally form liposomes that are incapable of filtration sterilization can be filter sterilized if the phospholipid is combined with an anti-inflammatory pharmaceutical including an NSAID, a COX-2 inhibitor, or similar anti-inflammatory agents or mixtures or combinations thereof. These sterile filtered compositions are then capable of administration orally, topically, intravenously, intra-arterially, or directly into a tissue or injury site for the prevention, treatment or amelioration of inflammation, pain, fever, or related symptoms. Phospholipid/anti-inflammatory pharmaceutical (PL-AIP) compositions are known to have enhanced efficacy in animals models for the prevention, treatment or amelioration of inflammation, pain, fever, or related symptoms. The inventors believe that phospholipids, in the absence of an anti-inflammatory pharmaceutical, exist as liposomes of a size that renders them either totally non-filterable or minimally filterable through a filter capable of generating a compositions considered sterile for medical applications. Once the anti-inflammatory pharmaceutical is added to a PL liposomal preparation with agitation, the inventors, without meaning to be tied to any specific explanation for the effect, believe that the particle size is reduced facilitating filtration. If the particles are of reduced size, then the anti-inflammatory pharmaceutical are thought to cause the particles to transition from multilamellar liposomes to either unilamellar liposomes or micellar particles or mixtures or combinations thereof. Alternatively, the addition of the anti-inflammatory pharmaceutical to a liposomal phospholipid preparation may render the liposomes more deformable so that they can pass through the pores of the filters having sufficiently small pore size to form sterile filtered compositions under appropriate extrusion pressures.

The compositions of this invention are ideally suited for pain management under situations where sterile administration is the preferred administration format. The sterile composition of this invention can be used in postoperative pain management, battle field pain management, accident pain management, or other pain management under emergency conditions without the significant side effects of alternative pain management medications such as opiates.

The present invention broadly relates to a composition including a phospholipid (PL) and an anti-inflammatory pharmaceutical (AIP), where the composition is capable of sterile filtration to form a filter sterilized PL-AIP composition.

The present invention broadly relates to a method of making a composition including a phospholipid (PL), such as a phosphatidylcholine (PC), and an anti-inflammatory pharmaceutical (AIP), such as a nonsteroidal, anti-inflammatory drug (NSAID), a COX-2 inhibitor, or mixtures or combinations thereof, that can be sterilized by filtration to form a sterile PL-AIP composition for administration by injection. The method includes the step of contacting a phospholipid with a buffer solution including an NSAID, a COX-2 inhibitor, or mixtures thereof at a pH range sufficient to facilitate filtration sterilization. The solution is then agitated for a time and at a temperature sufficient to form a PL-AIP formulation capable of filtration sterilization. The agitation time is generally between about 1 minute and about 60 minutes, preferably, between about 5 minutes to about 50 minutes, particularly, between about 10 minutes and 40 minutes, more particularly, between about 10 minutes and about 30 minutes and especiallybetween about 10 minutes and about 20 minutes. The temperature is generally ambient or room temperature, but temperature between about 0 and about 75° C. can be used as well provided that the components are stable at these temperatures. When heating is required, the agitated temperature is between ambient temperature and about 75° C. The resulting agitated formulation is then passed through a filter such as a membrane filter having a sufficiently small pore size under appropriate extrusion pressures to form a sterile PL-AIP composition. The extrusion pressures are dependent on the exact filter being used, but generally are between about atmospheric pressure and 14 bar or higher. These sterile PL-AIP formulations are then adjusted to a biological pH and stored as an injectable composition.

NSAIDs and COX-2 inhibitors are effective pain-relievers and anti-inflammatory agents that can be taken by mouth. However, in unconscious patients suffering from trauma to the head or other head injuries, health care professional must administer drugs via injection.

Phospholipid-anti-inflammatory pharmaceutical (PL-AIP) formulations are drug formulations that have fewer side effects, reduce GI toxicity, than regular NSAIDs or COX-2 inhibitors or mixtures thereof. Because PL-AIP formulations tend not to damage hydrophobic membranes or layer, PL-AIP formulations should be safer for patients needing such drugs for treatment of chronic conditions. PL-AIP formulations may also be absorbed faster across the blood-brain barrier than regular NSAIDs, because the PL component is similar to in nature to the hydrophobic character of the blood-brain barrier. Therefore, a method to make PL-AIP formulations that are sterile and can be administered intravenously, intra-arterially, intramuscularly or directly to a tissue or injury site would be useful for trauma patients being ventilated or for treating accident and battle field injuries.

Because of solubility limitations, there are only a few NSAIDs that are approved for injections, and none of them are complexed to PL. This new method allows most NSAIDs to be used parenterally when complexed to a PL producing fewer side effects, and may show faster absorption into the central nervous system, injury site or tissues site to which they are administered. We have demonstrated the preparation of sterile, filterable, injectable PL-AIP compositions such as PC-aspirin, PC-indomethacin, and PC-ibuprofen, where DPPC is dipalmitoylphosphatidylcholine.

Although sonication is the preferred agitation technique, other techniques such as high speed stirring, forcing the solution through a small nozzle at or near sonic velocities or other agitation techniques known to intimately mix components and then reduce the particles sized formed can be used as well.

Suitable phospholipids for use in this invention include, without limitation, a phospholipid of general formula: 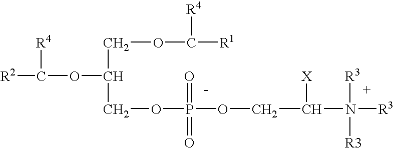
where R′ is H, OH or Cl and R is: (a) an alkyl group having 1 to 6 carbon atoms, optionally substituted with amino, alkylamino. dialkylamino or heterocyclyl, where the alkyl groups in alkylamino and dialkylamino substituents have 1 to 5 carbon atoms and are the same or different in the case of the dialkylamino substituted alkyl groups; (b) a halogen; (c) an arylthio, preferably chlorosubstituted; (d) a cycloalkylamino having 5 to 7 carbon atoms; or (e) a saturated five or six membered nitrogen containing heterocyclyl having 1 or 2 heteroatoms; and R₁ and R₂ are saturated or unsaturated substitutions ranging from 8 to 32 carbon atoms; R₃ is H or CH₃, and X is H or COOH; and R₄ is ═O or H₂. Mixtures and combinations of the zwitterionic phospholipids of the general formula and mixtures and combinations of NSAIDs can be used as well.

Exemplary examples of zwitterionic phospholipid of formula (II) include, without limitation, phosphatidylcholines such as phosphatidylcholine (PC), dipalmitoylphosphatidylcholine (DPPC), other disaturated phosphatidylcholines, phosphatidylethanolamines, phosphatidylinositol, phosphatidyl serines sphingomyelin or other ceramides, or various other zwitterionic phospholipids, phospholipid containing oils such as lecithin oils derived from soy beans, dimyristoyl phosphatidylcholine, distearoylphosphatidylcholine, dilinoleoylphosphatidylcholine (DLL-PC), dipalmitoylphosphatidylcholine (DPPC), soy phophatidylchloine (Soy-PC or PCs) and egg phosphatidycholine (Egg-PC or PCE). In DPPC, a saturated phospholipid, the saturated aliphatic substitution R₁ and R₂ are CH₃—(CH₂)₁₄, R₃ is CH₃ and X is H. In DLL-PC, an unsaturated phospholipid, R₁ and R₂ are CH₃—(CH₂)₄—CH═CH—CH₂—CH═CH═(CH₂)₇, R₃ is CH₃ and X is H. In Egg PC, which is a mixture of unsaturated phospholipids, R₁ primarily contains a saturated aliphatic substitution (e.g., palmitic or stearic acid), and R₂ is primarily an unsaturated aliphatic substitution (e.g., oleic or arachidonic acid). In Soy-PC, which in addition to the saturated phospholipids (palmitic acid and stearic acid) is a mixture of unsaturated phospholipids, [oleic acid, linoleic acid and linolenic acid]. The preferred zwitterionic phospholipid include, without limitation, dipalmitoyl phosphatidylcholine, phosphatidyl choline, or a mixture thereof.

Suitable NSAIDS include, without limitation, Propionic acid drugs such as Fenoprofen calcium (Nalfon.RTM.), Flurbiprofen (Ansaid .RTM.), Suprofen. Benoxaprofen, Ibuprofen (prescription Motrin .RTM.), Ibuprofen (200 mg. over the counter Nuprin, Motrin 1B .RTM.), Ketoprofen (Orduis, Oruvall .RTM.), Naproxen (Naprosyn .RTM.), Naproxen sodium (Aleve, Anaprox, Aflaxen .RTM.), Oxaprozin (Daypro .RTM.), or the like; Acetic acid drug such as Diclofenac sodium (Voltaren .RTM.), Diclofenac potassium (Cataflam .RTM.), Etodolac (Lodine .RTM.), Indomethacin (Indocin .RTM.), Ketorolac tromethamine (Acular, Toradol .RTM. intramuscular), Ketorolac (oral Toradol .RTM.), or the like; Ketone drugs such as Nabumetone (Relafen .RTM.), Sulindac (Clinoril .RTM.), Tolmetin sodium (Tolectin .RTM.). or the like; Fenamate drugs such as Meclofenamate sodium (Meclomen .RTM.), Mefenamic acid (Ponstel .RTM.), or the like; Oxicam drugs such as Piroxicam (Dolibid .RTM.), or the like; Salicylic acid drugs such as Diflunisal (Feldene .RTM.), Aspirin, or the like; Pyrazolin acid drugs such as Oxyphenbutazone (Tandearil .RTM.), Phenylbutazone (Butazolidin .RTM.), or the like; acetaminophen (Tylenol .RTM.), or the like, or mixtures or combinations thereof.

Suitable COX-2 inhibitors for using in this invention include, without limitation, celecoxib, meloxicam, diclofenac, meloxicam, piroxicam, or newly approved COX-2 inhibitors or mixtures or combinations thereof.

Suitable solvents for dissolving the phospholipid include, without limitation, chlorinated solvents such as chloroform, dichloromethane, trichloromethane, dichloroethane, trichloroethane, alkanes such as hexane, heptane, octane, or other solvents that dissolve phospholipids.

Generally, the weight ratio of NSAID to zwitterionic phospholipid is between about 1:0.01 and about 1:100, with ratios between about 1:0.02 and 1:50 being preferred and ratios between about 1:0.1 and 1:10 being particularly preferred and ratios between about 1:1 and about 1:5-being especially preferred. The effective amount of the NSAID for use in the composition of this invention ranges from about 1 mg per dose to about 1000 mg per dose depending on the NSAID and the phospholipid used in the composition, with doses between about 50 mg per dose to about 1000 mg per dose being preferred, doses of 83 mg per dose (for ASA), or about 100 mg per dose, of about 200 mg per dose, of about 400 mg per dose, of about 500 mg per dose, of about 600 mg per dose, of about 800 mg per dose and of about 1000 mg per dose being particularly preferred. A sufficient amount of phospholipid is generally an amount of phospholipid between about 0.1 mg per dose to about 5000 mg per dose, with amounts between about 1 mg per dose to 2500 mg per dose being preferred and amount between 2 mg per dose to about 250 mg per dose being particularly preferred and amounts between about 2 mg per dose and about 100 mg per does being especially preferred. Of course, the exact amount of NSA/D or COX-2 inhibitor in the PL-AIP compositions of this invention are adjusted to correspond to doses generally used for each of the NSAIDs and COX-2 inhibitors.

The associated complexes of this invention can be prepared according to the methods set forth in the following U.S. Pat. Nos. 5,955,451; 5,763,422; 5,260,287; 5,260,284; 5,134,129; 5,043,329; 5,032,464; 4,950,658 and 4,918,063, and co-pending U.S. patent application Ser. No. 10/433,454; incorporated herein by reference.

The compositions of the present invention can be in any desirable injectable form, including, without limitation, a suspension, a dispersion, a solution or any other injectable form of the PL-AIP formulations in a bio-compatible medium such as water. In this invention, a dispersion or suspension means a PL-AIP composition that passes through a filter of a sufficiently small size to produce a sterile composition.

### EXPERIMENTAL SECTION

General Methodology

In the following experiments, the phospholipid was dipalmitoyl phosphatidylcholine (DPPC) was used. The methodology started with dissolving a DPPC sample in a solvent, such as chloroform in glass vials. To these solvent solutions of DPPC were added tracer amounts of radiolabeled ³H-DPPC. The solvent was evaporated under nitrogen gas to form a phospholipid film. The phospholipid film was then resuspended in a buffer solution such as 2.5% sodium bicarbonate, pH 8.2, or 67 mM phosphate buffer having various pH values by sonication for a given period of time in a bath sonication. When making a PC-NSAID formulation, the NSAID was dissolved in the buffer solution prior to adding the buffer to the phospholipid film. After adding the NSAID buffer solution, the formulations were sonicated for a given period of time, generally, between about 10 to about 20 minutes, as noted. The resulting formulations were then forced through 0.2 μm membrane filters. The filtrate, as well as unfiltered material, were counted for tritium in a scintillation counter. Results are expressed as the percent of phospholipid that passed through the filter.

### Example 1

In this example, a 5 mM DPPC solution and a 5 mM DPPC/5 mM indomethacin (INDO) solution were filtered through a 0.2 μm membrane filter.

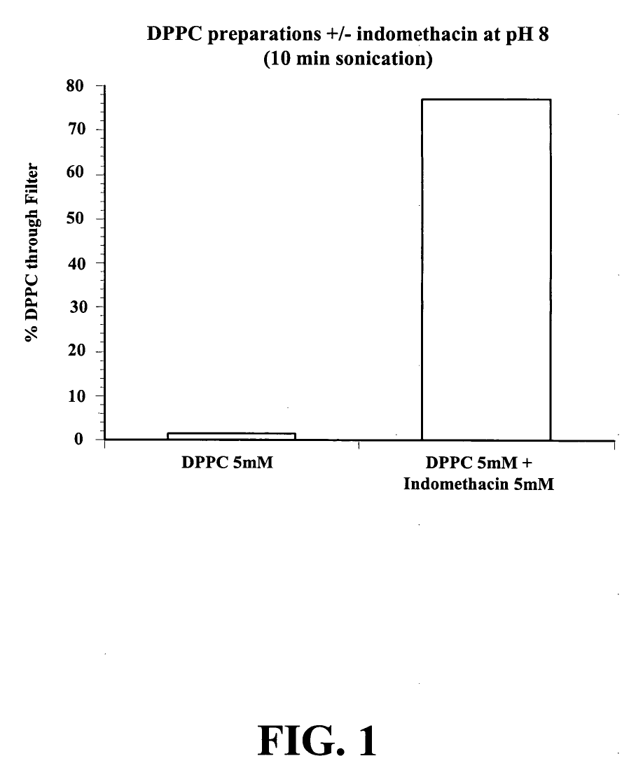

The solutions were prepared as described above in a 2.5% sodium bicarbonate buffer at pH 8. As shown in FIG. 1, the DPPC preparation did not pass through the filter (less then 2%). However, when complexed to INDO, the DPPC/INDO preparation easily passed through the filter (near 80%).

### Example 2

In this example, a DPPC solution, a DPPC/INDO solution and a DPPC/ibuprofen (IBU) were filtered through a 0.2 μm membrane filter.

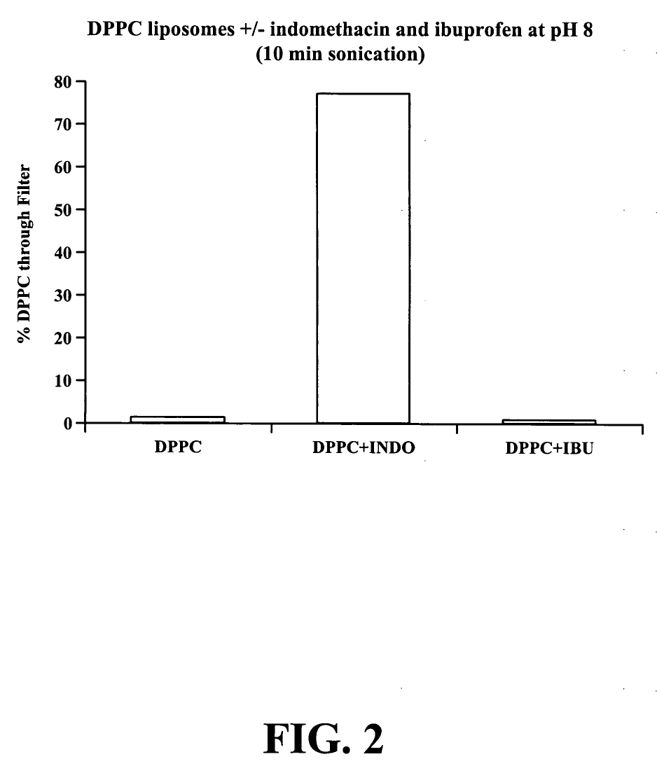

The DPPC/INDO and DPPC/IBU solutions were prepared using a 2.5% sodium bicarbonate buffer at pH 8. As shown in FIG. 2, again the DPPC preparation did not pass through the filter (less then 2%), while the DPPC/INDO preparation easily passed through the filter (near 80%). However, the DPPC/IBU preparation did pass through the filter (less than 1%).

### Example 3

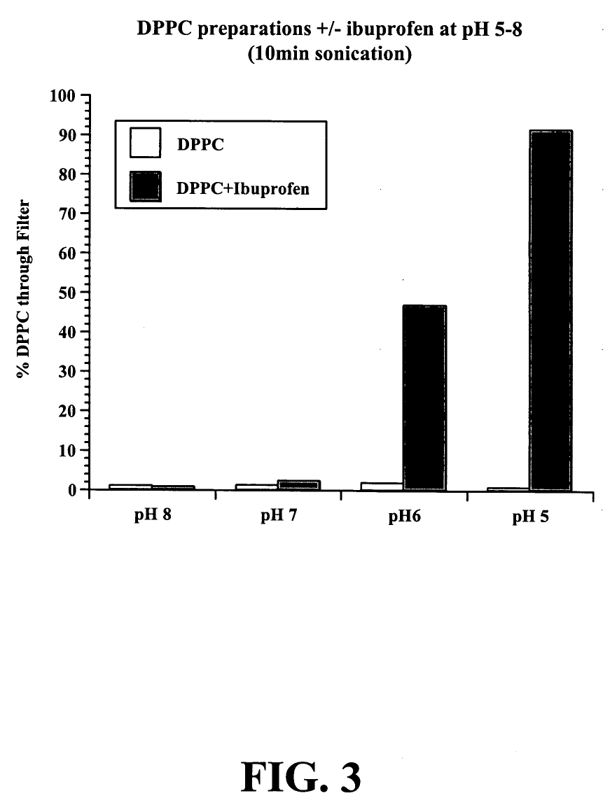

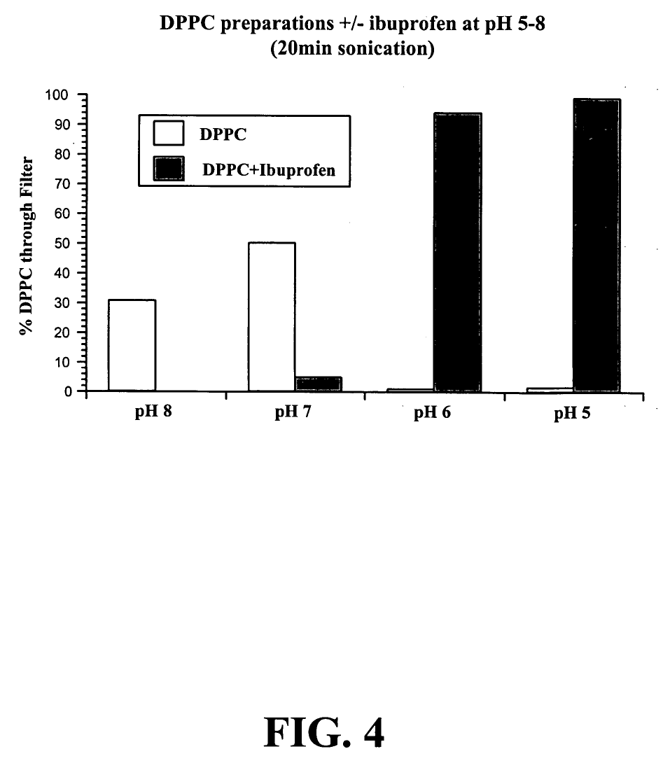

To test whether the combination of DPPC and ibuprofen (IBU) is affected by pH, a buffer system based on phosphate that can be adjusted over a wide range of pH values, was employed. DPPC preparations were formed in buffer at pH 5, 6, 7, or 8.2 and in the presence and absence of mBU. Samples were filtered after 10 and 20 minutes of sonication. As shown in FIGS. 3 and 4, at pH values greater than 6, DPPC/IBU solutions do not readily pass through the filter, but a pH values less than 7, the DPPC/IBU solution readily pass through the filter. The Figures also show that at sonication time also affects the percent of material that passes through the filter. At 10 minutes of sonication at pH 6, less than 50% of the DPPC/IBU solution passed through the filter, while at 20 minutes of sonication at pH 6, near 100% of the DPPC/IBU solution passed through the filter. At pH 5, nearly 100% of the DPPC/IBU solution passed through the filter.

### Example 4

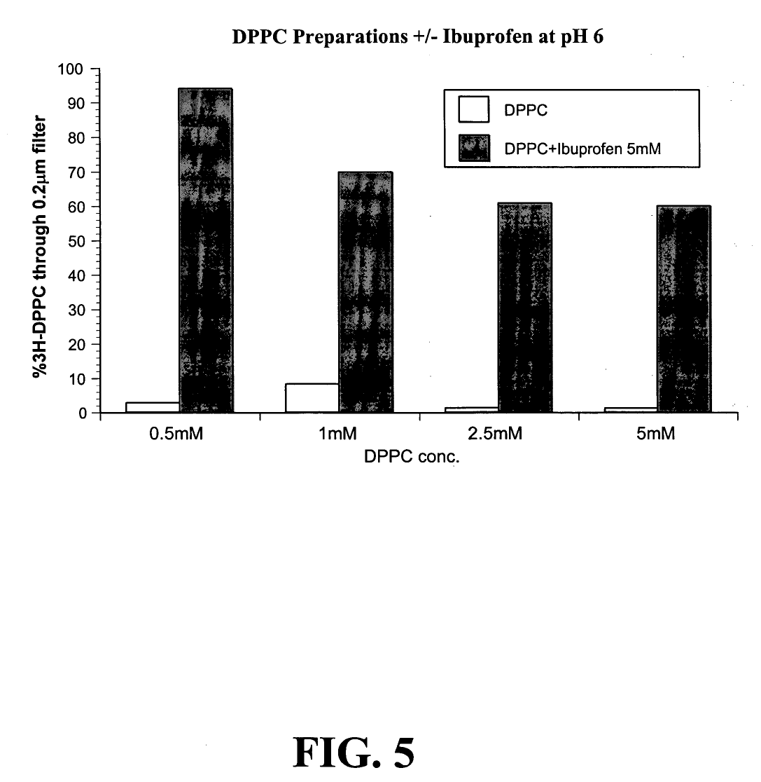

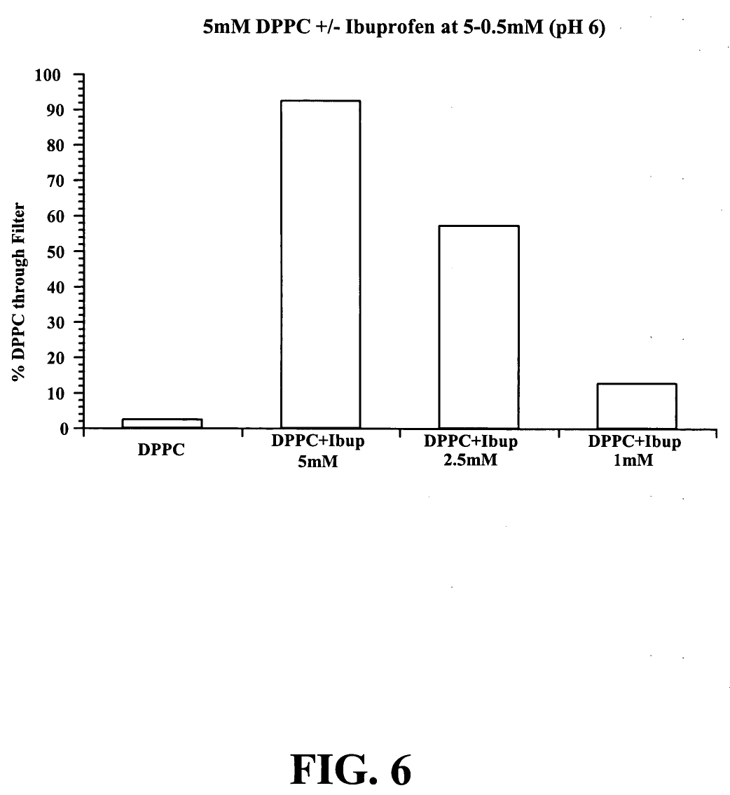

For the next two studies, the effect of altering the DPPC and ibuprofen (IBU) concentrations was examined. First, IBU concentration was held constant at 5 mM and DPPC concentration was varied from 0.5 to 5 mM. As shown in FIG. 5, almost none of the DPPC alone passed through the filter as before, but almost all of the DPPC/IBU preparations passed through the filter at all DPPC concentrations. Second, DPPC concentration was held constant at 5 mM and the IBU concentration was varied from 1 mM to 5 mM. As shown in FIG. 6, there was a clear dose-dependent reduction of the ability of the complex to pass through the filter as the amount of IBU was reduced. These results suggest that there is a critical IBU concentration needed to facilitate the filtration of a DPPC/IBU preparation. The critical concentration for IBU appears to be near equi-molar concentrations.

### Example 5

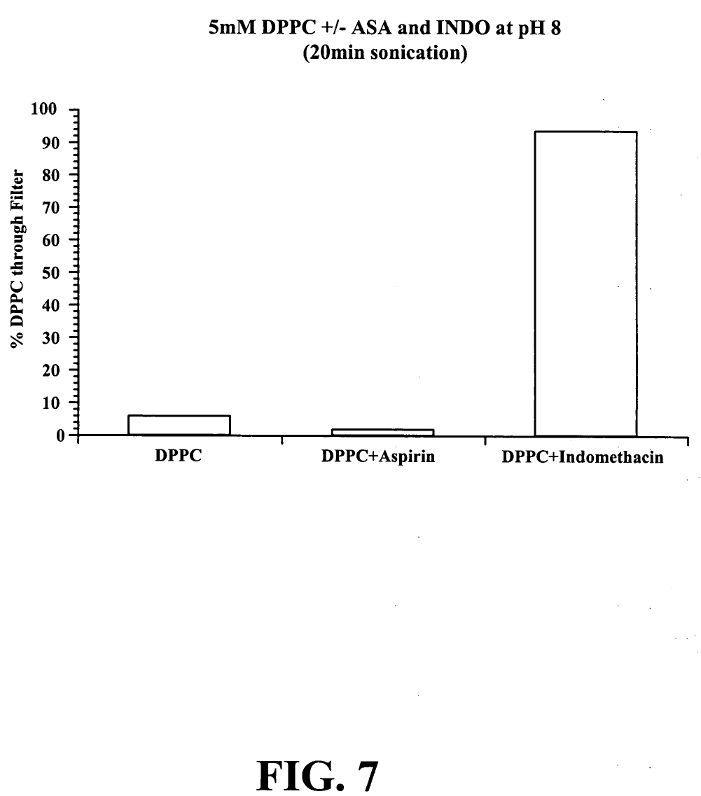

Another NSAID to be examined for complex formation with DPPC and filterabiliyt was aspirin (ASA). DPPC/ASA and DPPC/INDO (for comparison purposes) preparations were prepared in a 2.5% sodium bicarbonate buffer at pH 8 at equimolar concentrations and sonicated for 20 minutes. As shown in FIG. 7, the DPPC/ASA preparation did not pass through the filter at all, while the DPPC/INDO preparation did pass as usual.

### Example 6

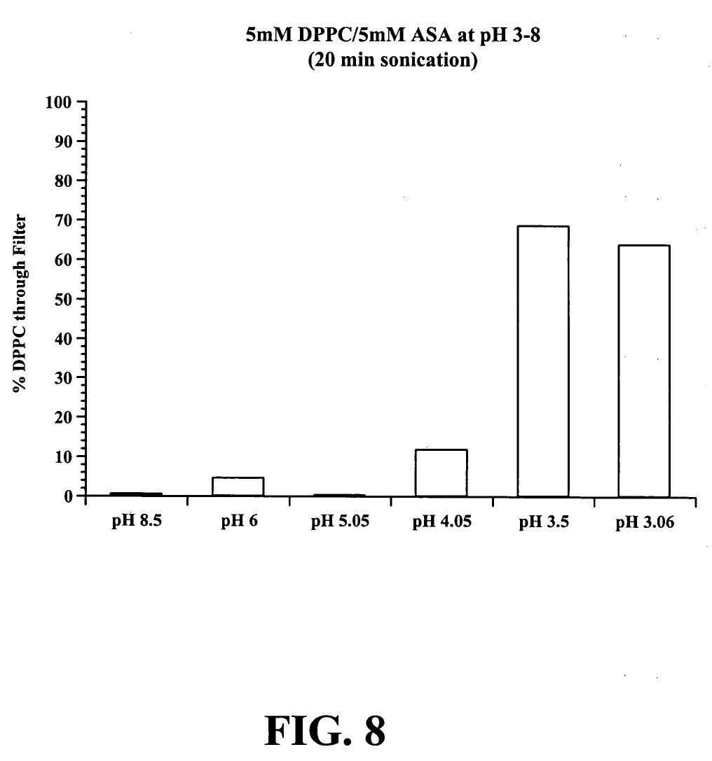

To determine whether the DPPC/ASA complex might pass through the filter at another pH, DPPC/ASA preparations were prepared using phosphate buffers having different pH values. The preparations all contained 5 mM DPPC and 5 mM ASA and were sonicated for 20 minutes at pH values between 3 and 8. As shown in FIG. 8, at pH 3.5 and below, the DPPC/ASA preparations readily passed through the filter.

### CONCLUSION

The above examples demonstrate that phospholipids such as a PC can be filter-sterilized when pre-complexed or pre-associated with an anti-inflammatory pharmaceutical to form filterable phospholipid/anti-inflammatory pharmaceutical (PL-AIP) preparations, where the anti-inflammatory pharmaceutical includes NSAIDs, COX-2 inhibitors or mixtures thereof; provided, of course, that the pH is adjusted to a value that permits the preparations to pass through the filters and that agitation is continued for a time sufficient to form filterable compositions. Such filter-sterilized PL-AIP are then suitable for intravenous administration, intra-arterial administration, topical administration or direct administration into veins, arteries, tissues, and injuries, where the pH of the filtrate containing the PC-AIP particles will then be adjusted to 7.4 prior to parenteral administration

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

1. A filter sterilized composition comprising a phospholipid and an anti-inflammatory pharmaceutical capable of passing through a sterilizing filter having a pore size sufficiently small to result in a sterile composition capable of administration by injection.

2. The composition of claim 1, wherein the sufficiently small pore size is about 0.22 μm or less.

3. The composition of claim 1, wherein the phospholipid is a compound having the following formula: 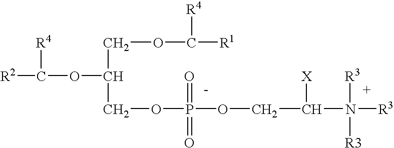
where R′ is H, OH or Cl and R is: (a) an alkyl group having 1 to 6 carbon atoms, optionally substituted with amino, alkylamino. dialkylamino or heterocyclyl, where the alkyl groups in alkylamino and dialkylamino substituents have 1 to 5 carbon atoms and are the same or different in the case of the dialkylamino substituted alkyl groups; (b) a halogen; (c) an arylthio, preferably chlorosubstituted; (d) a cycloalkylamino having 5 to 7 carbon atoms; or (e) a saturated five or six membered nitrogen containing heterocyclyl having 1 or 2 heteroatoms; and R₁ and R₂ are saturated or unsaturated substitutions ranging from 8 to 32 carbon atoms; R₃ is H or CH₃, and X is H or COOH; and R₄ is ═O or H₂, and mixtures and combinations thereof.

4. The composition of claim 1, wherein the phospholipid is selected from the group consisting of phosphatidyl choline (PC), dipalmitoylphosphatidylcholine (DPPC), other disaturated phosphatidylcholines, phosphatidyl ethanolamines, phosphatidylinositol, phosphatidyl serines sphingomyelin or other ceramides, other zwitterionic phospholipids, phospholipid containing oils such as lecithin oils derived from soy beans, dimyristoyl phosphatidylcholine, distearoyl phosphatidylcholine, dilinoleoyl-phosphatidylcholine (DLL-PC), dipalmitoyl-phosphatidylcholine (DPPC), soy phophatidylchloine (Soy-PC or PC_{S}) and egg phosphatidycholine (Egg-PC or PCE). In DPPC, a saturated phospholipid, the saturated aliphatic substitution R_{1 and R}₂ are CH₃—(CH₂)₁₄, R₃ is CH₃ and X is H. In DLL-PC, an unsaturated phospholipid, R_{1 and R}₂ are CH₃—(CH₂)₄—CH═CH—CH₂—CH═CH—(CH₂)₇, R₃ is CH₃ and X is H. In Egg PC, which is a mixture of unsaturated phospholipids, R₁ primarily contains a saturated aliphatic substitution (e.g., palmitic or stearic acid), and R₂ is primarily an unsaturated aliphatic substitution (e.g., oleic or arachidonic acid). In Soy-PC, which in addition to the saturated phospholipids (palmitic acid and stearic acid) is a mixture of unsaturated phospholipids, [oleic acid, linoleic acid and linolenic acid], and mixtures or combinations thereof.

5. The composition of claim 1, wherein the phospholipid is selected from the group consisting of dipalmitoyl phosphatidylcholine, phosphatidyl choline, and mixtures or combinations thereof.

6. The composition of claim 1, wherein the anti-inflammatory pharmaceutical is selected from the group consisting of a nonsteroidal, anti-inflammatory drug (NSAID), a cyclooxygenase 2 (COX-2) inhibitor and mixtures or combinations thereof.

7. The composition of claim 6, wherein the NSAID is selected from the group consisting of: Propionic acid drugs including Fenoprofen calcium (Nalfon.RTM.), Flurbiprofen (Ansaid .RTM.), Suprofen. Benoxaprofen, Ibuprofen (prescription Motrin .RTM.), Ibuprofen (200 mg. over the counter Nuprin, Motrin 1B .RTM.), Ketoprofen (Orduis, Oruvall .RTM.), Naproxen (Naprosyn .RTM.), Naproxen sodium (Aleve, Anaprox, Aflaxen .RTM.), and Oxaprozin (Daypro .RTM.); Acetic acid drugs including sodium (Voltaren .RTM.), Diclofenac potassium (Cataflam .RTM.), Etodolac (Lodine .RTM.), Indomethacin (Indocin .RTM.), Ketorolac tromethamine (Acular, Toradol .RTM. intramuscular), and Ketorolac (oral Toradol .RTM.); Ketone drugs including Nabumetone (Relafen .RTM.), Sulindac (Clinoril .RTM.), and Tolmetin sodium (Tolectin .RTM.); Fenamate drugs including Meclofenamate sodium (Meclomen .RTM.), Mefenamic acid (Ponstel .RTM.), or the like; Oxicam drugs such as Piroxicam (Dolibid .RTM.), or the like; Salicylic acid drugs such as Diflunisal (Feldene .RTM.), and Aspirin; Pyrazolin acid drugs including Oxyphenbutazone (Tandearil .RTM.), and Phenylbutazone (Butazolidin .RTM.); acetaminophen (Tylenol .RTM.), and mixtures or combinations thereof.

8. The composition of claim 6, wherein the COX-2 inhibitor is selected from the group consisting of celecoxib, meloxicam, diclofenac, meloxicam, piroxicam, or newly approved COX-2 inhibitors or mixtures or combinations thereof.

9. The composition of claim 1, where the composition comprises an associated complex of the phospholipid and the anti-inflammatory pharmaceutical.

10. A filter sterilized composition comprising a phospholipid and an anti-inflammatory pharmaceutical capable of passing through a sterilizing filter having a pore size sufficiently small to result in a sterile composition capable of administration by injection for pain management before, during and after an operation.

11. A method for making sterile composition comprising the steps of:
contacting a phospholipid and an anti-inflammatory pharmaceutical in a buffer under agitating conditions at a pH sufficient to promote filter sterilization of the composition; and
passing the agitated composition through a filter to produce a filter sterilized phospholipid/anti-inflammatory pharmaceutical composition,
where the filter includes pores having a size sufficiently small size to result in the filter sterilized phospholipid/anti-inflammatory pharmaceutical composition suitable of direct injection in an animal including a human body.

12. The method of claim 11, further comprising the step of:
adjusting the filter sterilized phospholipid/anti-inflammatory pharmaceutical composition to a physiological pH.

13. The method of claim 11, wherein the direct injection is selected from the group consisting of intravenous injection, intra-arterial injection, intramuscular injection, injection directly into a tissue site, injection directly into an injury site and injection according to an injection protocol including one or more intravenous injections, intra-arterial injections, intramuscular injections, injections directly into a tissue site, injections directly into an injury site.

14. A method for preparing a sterile filtration anti-inflammatory pharmaceutical composition comprising the steps of
dissolving a phospholipid (PL) in a solvent to form a PL solution,
removing the solvent from the PL solution to form a phospholipid film;
suspending the PL in the PL film in an aqueous solution of an anti-inflammatory pharmaceutical (AIP) having an operable pH with agitation, at a temperature and for a time sufficient to form to form an aqueous PL-AIP composition capable filter sterilization; and
passing or extruding the PL-AIP composition through a filter having a pore size sufficiently small to produce a sterile filtered PL-AIP composition.

15. The method of claim 14, further comprising the step of:
adjusting the PL-AIP composition to a physiological pH producing a composition suitable for direct injection in to an animal including a human body.

16. The method of claim 14, wherein the direct injection is selected from the group consisting of intravenous injection, intra-arterial injection, intramuscular injection, injection directly into a tissue site, injection directly into an injury site and injection according to an injection protocol including one or more intravenous injections, intra-arterial injections, intramuscular injections, injections directly into a tissue site, injections directly into an injury site.

17. The method of claim 14, wherein the operable pH is at or near a pKₐ value of the AIP or at a pH value sufficient for the PL-AIP composition to pass through the filter.

18. The method of claim 14, wherein the PL-NSAID composition comprises PL-AIP unilamellar liposomes, micelles or mixtures or combinations thereof, where the liposomes and micelles are capable of passing through the sterilizing filter.

19. The method of claim 14, wherein:
the composition comprises an associated complex of a phospholipid and an anti-inflammatory pharmaceutical,
the pore size is about 0.22 μm or less,
the phospholipid is a compound having the following formula: 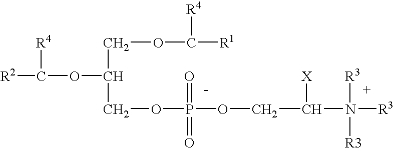
where R′ is H, OH or Cl and R is: (a) an alkyl group having 1 to 6 carbon atoms, optionally substituted with amino, alkylamino. dialkylamino or heterocyclyl, where the alkyl groups in alkylamino and dialkylamino substituents have 1 to 5 carbon atoms and are the same or different in the case of the dialkylamino substituted alkyl groups; (b) a halogen; (c) an arylthio, preferably chlorosubstituted; (d) a cycloalkylamino having 5 to 7 carbon atoms; or (e) a saturated five or six membered nitrogen containing heterocyclyl having 1 or 2 heteroatoms; and R_{1 and R}₂ are saturated or unsaturated substitutions ranging from 8 to 32 carbon atoms; R₃ is H or CH₃, and X is H or COOH; and R₄ is =0 or H₂, and mixtures and combinations thereof, and
the anti-inflammatory pharmaceutical is selected from the group consisting of a nonsteroidal, anti-inflammatory pharmaceutical drug (NSAID), a COX-2 inhibitor and mixtures or combinations thereof.

20. The method of claim 19, wherein:
the NSAID is selected from the group consisting of: Propionic acid drugs including Fenoprofen calcium (Nalfon.RTM.), Flurbiprofen (Ansaid .RTM.), Suprofen. Benoxaprofen, Ibuprofen (prescription Motrin .RTM.), Ibuprofen (200 mg. over the counter Nuprin, Motrin 1B .RTM.), Ketoprofen (Orduis, Oruvall .RTM.), Naproxen (Naprosyn .RTM.), Naproxen sodium (Aleve, Anaprox, Aflaxen .RTM.), and Oxaprozin (Daypro .RTM.); Acetic acid drugs including sodium (Voltaren .RTM.), Diclofenac potassium (Cataflam .RTM.), Etodolac (Lodine .RTM.), Indomethacin (Indocin .RTM.), Ketorolac tromethamine (Acular, Toradol .RTM. intramuscular), and Ketorolac (oral Toradol .RTM.); Ketone drugs including Nabumetone (Relafen .RTM.), Sulindac (Clinoril .RTM.), and Tolmetin sodium (Tolectin .RTM.); Fenamate drugs including Meclofenamate sodium (Meclomen .RTM.), Mefenamic acid (Ponstel .RTM.), or the like; Oxicam drugs such as Piroxicam (Dolibid .RTM.), or the like; Salicylic acid drugs such as Diflunisal (Feldene .RTM.), and Aspirin; Pyrazolin acid drugs including Oxyphenbutazone (Tandearil .RTM.), and Phenylbutazone (Butazolidin .RTM.); acetaminophen (Tylenol .RTM.), and mixtures or combinations thereof, and
the COX-2 inhibitor is selected from the group consisting of celecoxib, meloxicam, diclofenac, meloxicam, piroxicam, or newly approved COX-2 inhibitors or mixtures or combinations thereof.

21. A method comprising the steps of:
administering a pharmaceutically effective amount of a filter sterilized phospholipid/anti-inflammatory pharmaceutical (PL-AIP) composition to an animal including a human to ameliorate inflammation, pain, fever, and other related symptoms.

22. The method of claim 21, wherein the administering step is selected from the group consisting orally administering, topically administering, intravenously administering, intra-arterially administering and directly administering into a tissue site.

23. The method of claim 21, wherein the administering step comprises a single administering step, periodic administering steps, intermittent administering step, or an administering protocol.

24. The method of claim 21 wherein the administering protocol includes one or more orally administering steps, topically administering steps, intravenously administering steps, intra-arterially administering steps or direct into a tissue site administering steps.

25. A method of treating injuries to tissues including neurons comprising the step of:
administering a pharmaceutically effective amount of a filter sterilized phospholipid/anti-inflammatory pharmaceutical (PL-AIP) composition to an animal including a human to ameliorate inflammation, pain, fever, and other related symptoms associated with an injury to tissue including neurons.

26. The method of claim 25, wherein the tissue including neurons is selected from the group consisting of a spinal cord, a central nervous system, a peripheral nervous system, and mixtures or combinations thereof.

27. A method of treating field injuries including accident and combat injuries comprising the step of:
administering a pharmaceutically effective amount of a filter sterilized phospholipid/anti-inflammatory pharmaceutical (PL-AIP) composition to an animal including a human to ameliorate inflammation, pain, fever, and other related symptoms associated an accident or combat induced injury, while preventing ulceration of the injury or to maintain the integrity of hydrophobic membranes and/or layers associated with the injury.

28. A method of pain management comprising the step of:
administering a pharmaceutically effective amount of a filter sterilized phospholipid/anti-inflammatory pharmaceutical (PL-AIP) composition to an animal including a human to ameliorate inflammation, pain and other related symptoms of a medical condition requiring pain management via direct injection.

29. The method of claim 28, wherein the medical condition is a postoperative condition.
